(19) United States
(12) Patent Application Publication
(10) Pub. No.: US 2004/0067485 A1
(43) Pub. Date: Apr. 8, 2004

(54) Nanoparticles

(75) Inventors: Eric L. Mayes, Bristol (GB); Kim K.W. Wong, Bristol (GB); Barnaby Warne, Bristol (GB); James M. Houlihan, Bristol (GB)

Correspondence Address: BURNS, DOANE, SWECKER & MATHIS, L.L.P., P.O. Box 1404, Alexandria, VA, 22313-1404, US

(73) Assignee: Nanomagnetics Limited

(21) Appl. No.: 10/375,175

(22) Filed: Feb. 28, 2003

(30) Foreign Application Priority Data
Oct. 4, 2002 (GB) 0223149.6

(51) Int. Cl. C12Q 1/70

(52) U.S. Cl. 435/5; 435/7.5; 436/524; 436/525

(57) ABSTRACT

A method for the synthesis of a semiconductor nanoparticle within a protein template. The semiconductor is selected from cadmium selenide, cadmium telluride, zinc selenide, zinc sulfide and zinc telluride. The process comprises forming a reaction mixture by combining in a liquid medium a cation source selected from cadmium or zinc ions and an anion source selected from sulfur, selenium or tellurium ions in the presence of a source of a protein which is capable of acting as a template for the formation of nanoparticles and maintaining said liquid medium at a temperature of at least 24° C. for a time sufficient to permit nanoparticle formation within the protein template, with the proviso that when the cation source is cadmium, the anion source is not sulfur.

### FIELD OF THE INVENTION

This invention relates to protein-encapsulated semiconductor nanoparticles, in particular nanoparticles of a semiconductor material which is selected from cadmium selenide, cadmium telluride, zinc selenide, zinc sulfide and zinc telluride and mixtures thereof. The invention also relates to methods of making such encapsulated semiconductor particles. The invention also extends to the semiconductor nanoparticles obtained by removing the protein encapsulant, and to the methods for obtaining such materials. The encapsulated nanoparticles and nanoparticles treated to remove the encapsulant find a variety of uses as discussed herein.

### BACKGROUND OF THE INVENTION

Photo-active semiconductor nano-sized materials (SCNM) possess unique light emission and absorption characteristics which are determined by their crystal size and composition. Accordingly, by modifying the crystalline form of SCNM an array of discrete band gap energies and consequently emission spectra can be produced. One such class of semi-conductor particles are known as Quantum Dots. These particles present the opportunity to construct multiple-colour luminescent systems. They also exhibit a relatively high degree of photo-stability compared to conventional dyes.

On account of their unique properties, SCNM have found application in a variety of photoelectronic and biological technologies. For example, the use of cadmium selenide and zinc sulphide quantum dots in tracking cancer cells has recently been described (Parak W J et.al. 2002 Adv. Mater, 14(2) pages 882-885. Parak W J et.al. (2002 Chem. Mater., 14 pages 2113-2118) also report quantum dot particles coated with silanes as vehicles for nucleic acid probes. Further, Huynh W. U. et.al. 2002 (Science, 295 pages 2425-2427) have described the construction of fabricated solar cells comprising cadmium selenide nano-rods.

The production and characterization of SCNM in solution by chemical synthesis procedures is well documented, for example by Revaprasadu N. et.al. 1999 (Chem. Comm., 16 pages 1573-1574).

Previous literature describes the synthesis of materials in ferritin templates: iron and manganese oxides (Meldrum F. et.al. 1995 J. Inorg. Biochem., 58 (1) pages 5968); magnetite (Wong K. K. W. et.al. 1998 Chem. Mater., 10 pages 279-285) and cadmium sulphide (Wong K. K. W. & S Mann 1996 Adv. Mater., 8 (11) pages 928932).

The present invention concerns the synthesis of nanoparticles within protein templates. Whilst the synthesis of cadmium sulfide nanoparticles within a ferritin template has already been reported by Wong et al. supra, the present inventors have not hitherto been able to replicate the synthetic procedure described for the synthesis of other SCNM because it was found that SC particles did not form within the protein template but precipitated in the solution outside the protein. The present inventors have identified a modification of the Wong procedure which, surprisingly, enables the production of protein encapsulated semiconductor nanoparticles other than cadmium sulfide.

### SUMMARY OF THE INVENTION

In accordance with a first aspect of the present invention, there is provided a method for the synthesis of a semiconductor nanoparticle within a protein template, wherein the semiconductor is selected from cadmium selenide, cadmium telluride, zinc selenide, zinc sulfide and zinc telluride, which process comprises forming a reaction mixture by combining in a liquid medium a cation source selected from cadmium or zinc ions and an anion source selected from sulfur, selenium or tellurium ions in the presence of a source of a protein which is capable of acting as a template for the formation of nanoparticles and maintaining the liquid medium at a temperature of at least 24° C. for a time sufficient to permit nanoparticle formation within the protein template, with the proviso that when the cation source is cadmium, the anion source is not sulfur.

According to a second aspect of the present invention, there is provided a protein-encapsulated semi-conductor nanoparticle obtainable by the process of the first aspect of the invention, wherein said semiconductor is selected from CdSe, CdTe, ZnSe, ZnS or ZnTe.

According to a third aspect of the present invention there is provided a semi-conductor nanoparticle selected from CdSe, CdTe, ZnSe, ZnS or ZnTe obtained by treating or removing the protein encapsulating material from the protein-encapsulated semi-conductor nanoparticles of the second aspect of the present invention.

According to a fourth aspect of the present invention, there is provided a protein-encapsulated semi-conductor nanoparticle, wherein said semiconductor is selected from CdSe, CdTe, ZnSe, ZnS or ZnTe.

### DETAILED DESCRIPTION OF THE INVENTION

The method of the first aspect of the present invention is based on the finding that the process of Wong et al. may be adapted for the production of semiconductor particles other than cadmium sulfide by raising the temperature of the reaction mixture which contains the selected cation and anion sources and the template protein source above ambient temperature, for example to a temperature of at least 24° C.

The synthesis of SC nanoparticles within a protein template offers several advantages over conventional techniques. For instance, by selection of a particular protein species, the size of the core material, in particular a SCNM crystal, can be controlled. The protein shell also provides a means for facilitating the dispersion of the material encapsulated by the protein. Further, it conveniently provides a means for the attachment of ligands, particularly biological ligands, to SCNM by virtue of the exterior aspect of the protein shell.

The cation source is preferably a salt of cadmium or zinc, for example acetate, nitrate or sulphate salts may be used.

The anion source is preferably a salt, for example a sodium salt, of sulfur, selenium or tellurium. The acid forms, hydrogen sulphide, hydrogen telluride or hydrogen selenide may also be used.

As mentioned previously, the protein source used in the invention is one which is capable of acting as a template for the formation of nanoparticles. More particularly, the protein source should be capable of forming a structure (which may be an assembly of protein molecules) which can accommodate the synthesis of SCNM material therein and which will at least partially surround the formed core semiconductor nanoparticles.

The present invention preferably makes use of the iron storage protein, ferritin. Natural ferritin has a molecular weight of 450 kD and is utilised in iron metabolism throughout living species and its structure is highly conserved among them. It consists of 24 subunits which self-assemble to provide a hollow shell roughly 12 nm in outer diameter. It has an 8 nm diameter cavity which normally stores 4500 iron(III) atoms in the form of paramagnetic ferrihydrite. However, this ferrihydrite can be removed to leave a ferritin unit which is devoid of ferrihydrite and which is termed “apoferritin”. The subunits in ferritin pack tightly; there are, however, channels into the cavity at the 3-fold and 4-fold axes. The presently preferred macromolecule for use in the invention is the apoferritin protein which has a cavity of the order of 8 nm in diameter. The SCNM to be accommodated within this protein will have a diameter up to about 15 nm in diameter, as the protein can stretch to accommodate a larger particle than one 8 nm in diameter.

Ferritin can be found naturally in vertebrates, invertebrates, plants, fungi, yeasts, bacteria. It can also be produced synthetically through recombinant techniques. Such synthetic forms may be identical to the natural forms, although it is also possible to synthesise mutant forms which will still retain the essential characteristic of being able to act as a template for the formation of nanoparticles and accommodate a nanoparticle within its internal cavity. The use of all such natural and synthetic forms of ferritin is contemplated within the present invention.

Thus, in a preferred embodiment of the first aspect of the invention, the template protein is apoferritin. However, the ordinary addressee will appreciate that other protein systems capable of supporting a variety of particulate morphologies could be employed, such as DPS and other members of the ferritin family, viruses, bacteriophages, flagellar LP rings, microtubules which are tubular proteins, formed from αβ-tubulins, and have an outer diameter of about 25 nm and a length of several micrometres and chaperonins. DPS, in particular is a ferritin homologue, dodecamer DNA protection protein comprising a hollow core and pores in the three-fold axis. Flagellar LP rings are ring-shaped structures having an inner diameter of approximately 13 nm and outer diameter of approximately 20 nm. They can be induced to pack into well-ordered arrays extending over several microns, approximately 13 nm thick. At more dilute concentrations, dimers can form that are 26 mm thick.

Normally, the semiconductor nanoparticles of the invention will have all of their dimensions in the nano size range, typically at least 1 nm and no greater than 100 nm, preferably no greater than 50 nm and more preferably no greater than 20 nm. Preferred semiconductor nanoparticles of the invention are substantially spheroidal having a diameter in the range 1-100 nm. However the present invention also extends to semiconductor particles which have one dimension which is not within the nanosize range, as for example, the particles formed using microtubules which are tubular proteins, formed from αβ-tubulins, and have an outer diameter of about 25 nm and a length of several micrometres.

As previously noted, in the first process aspect of the present invention, the cation and anion sources are combined in a liquid medium. The liquid medium may be regarded as a “solution” in the sense that the components thereof are generally regarded as being solubilized, although such solutions can also be regarded as colloidal suspensions. The predominant component of the liquid medium is preferably water, although a percentage of one or more water-miscible solvents may also be present such as tetrahydrofuran or ethanol. For example tetrahydrofuran or other water miscible solvents may be present in a total amount of up to 50% by weight. The percentage of water-miscible solvents in the liquid medium is preferably less than 25% by weight, more preferably less than 10% by weight.

In the process of the present invention, it is preferred that the cation source and the anion source are added in incremental quantities to a solution of the protein source. For example the cation and anion sources may be added in sufficient amounts to provide more than 1 atom of the cation and anion sources per protein template per iteration, preferably greater than 20 atoms of the cation and anion sources per protein template per iteration. The cation and anion sources may be added in sufficient amounts to provide fewer than 200 atoms of the cation and anion sources per protein template per iteration, preferably fewer than 100 atoms of the cation and anion sources per protein template per iteration. In a preferred embodiment of the invention the cation and anion sources may be added in sufficient amounts to provide about 50 atoms of the cation and anion sources per protein template per iteration. These low concentrations may be achieved by successive dilutions of solutions containing the cation and anion sources.

The cation and anion sources should be added to the reaction mixture under inert conditions such as nitrogen or argon gas.

The preferred stoichiometric ratio of said cation source to the anion source in the reaction mixture formed is preferably no greater than two.

The reaction mixture may be formed at a temperature below the preferred temperature at which the nanoparticles are allowed to form and then raised to that temperature. Alternatively, the source of protein to which the source of anions and cations is to added may be held at a temperature of at least 24° C. and the sources of cation and anions added thereto. This latter procedure is preferred and may involve a series of incremental additions of cations and anions until all the ions have been taken up by the protein. The reaction may be monitored for example by X-ray fluorescence, energy dispersive X-ray analysis or atomic absorption spectroscopy.

Proteins can generally withstand temperatures of up to 70° C. before they lose their tertiary structure. Thus, in the present invention the temperature of reaction may range up to about 70° C. Preferably, the reaction temperature is maintained between 25 and 45° C. In a preferred embodiment of the present invention the reaction temperature is about 30° C.

The reaction mixture is maintained at the reaction temperature of at least 24° C. for a time sufficient to permit nanoparticle formation. This may be a time of between 15 and 120 minutes, preferably about 60 minutes.

Once the reaction is complete, residual component salts may be removed by conventional means such as dialysis or by size fractionation. The synthesised particles are then subject to ultracentrifugation and/or filtration to generate a dispersion of protein encapsulated nanoparticles characterised by their limited inter-particular size variation and mono-dispersity.

In addition, the application of filtration steps in the synthesis procedure may be utilised in order to promote the mono-dispersity of the solution. Thus, surprisingly, it has been found that size fractionation of either the initial encapsulating protein source prior to synthesis reactions and/or the synthesised encapsulated nanoparticles assists in generating a stable mono-disperse solution. For example, using membrane filtration it has been found that the size of the filtration pore can be several orders of magnitude higher than that of the diameter of the protein shell. In particular, we have found that filters with pore sizes of up to 0.2 μm will enhance the mono-dispersity of apoferritin-filled SCNM particles. More preferably, the filter size will be about 0.1 μm. In a preferred embodiment of the invention, the particles are filtered after synthesis. Further details of the use of a filtration step in the process of the invention are described below.

In an embodiment of the invention the protein shell may be treated or removed. For example, the protein shell may be treated to remove the protein shell and leave a core nanoparticle. Removal of the protein shell in this way may be accomplished, for example, by enzymatic degradation or pH denaturation. In particular, the protein may be digested using proteases or by adjusting the pH of the solution to a value outside the range at which the protein is stable, for example below about pH 4.0 or above about pH 9.0. Alternatively, the protein shell may be treated to leave a residue surrounding the nanoparticle core. For example, the protein shell may be carbonised by subjecting the protein encapsulated semiconductor nanoparticles to an elevated temperature, for example between about 300 and 450° C., sufficient to convert the protein to a carbonaceous material, or it may be fixed using glutaraldehyde.

In another embodiment of the invention, the protein encapsulating shell may be further treated, either before or after nanoparticle formation, to attach biological ligands to the outside of the shell. The ordinary addressee will understand that a variety of ligands such as antibodies or derivatives thereof, receptor molecules, opsonins, etc. may be attached to the surface of the protein shell. Preferably the ligands will be antibodies, or derivatives thereof such as ScFv fragments. Further, a variety of protocols are available for the conjugation of binding moieties to the surface of the protein (Wong S. S. 1993 “Chemistry of protein conjugation and cross-linking” CRC Press) and, in particular, biotinylation and avidinylation of ferritin have been described (Li M. et.al. 1999 Chem. Mater., 11 pages 23-26; Bayer E. A. et.al. 1976 J. Histochem. & Cytochem., 24 (8) pages 933-939).

As defined above, the present invention also provides, as a second aspect, a protein encapsulated semi-conductor nanoparticle which may be obtainable by the process described above, wherein said semiconductor is selected from CdSe, CdTe, ZnSe, ZnS or ZnTe.

The size of the nanoparticle of this second aspect of the invention will depend upon the protein structure which encapsulates it. The preferred nanoparticles of the invention, which are made using an apoferritin template are up to about 15 nm in diameter. As noted above, this is larger than the normal relaxed apoferritin cavity size, which is only 8 nm, but the ferritin structure is able to stretch somewhat to accommodate larger particles although the accommodated particle is usually of commensurate size with that of the relaxed apoferritin cavity. Generally, the nanoparticles are spheroidal in shape.

Although the preferred protein encapsulant is apoferritin, other proteins may be employed, as described above. The protein encapsulated semiconductor nanoparticles of this aspect of the invention may be provided as a population of such particles in either a dried form, for example as a lyophilised preparation, or in a suitable solution, normally aqueous. The particles in such a population should preferably have a high degree of monodispersity, i.e. the degree to which the size of the individual semiconductor nanoparticles varies within a composition of the invention. This variation, measured in terms of the largest nano-sized dimension, should normally be less than 20%, preferably less than 10% and most preferably less than 5%. For compositions in which the average size is relatively large, e.g. about 50 nm, it is preferred that the variation is at the lower end of the above ranges, whilst for relatively small particles, e.g. about 10 nm, the variation may be at the upper end of the above ranges. The sizes of the particles in accordance with the present invention can be measured using for example Transmission electron microscopy (TEM) (Jeol 2010; http://www.jeoleuro.com).

In addition, in an embodiment of the invention, the nanoparticles are provided in a form in which they are present as single unagglomerated particles, that is to say they are not present in clumps of particles, but rather are discrete particles which are spatially separated from each other in the composition. For example, at least 50% by weight of the nanoparticles, more preferably at least 70% by weight of the nanoparticles should be present as separate particles which are not agglomerated with another particle. One approach which has been found to be useful for the production of encapsulated nanoparticles which are unagglomerated is to subject a solution of the protein template (or a subunit thereof) to a microporous membrane filtration step prior to formation of the nanoparticles. Another approach is to subject a composition of the formed encapsulated nanoparticles to a microporous membrane filtration step. Membrane filters are well known structures which are distinguished from non-membrane filters by the fact that membranes have a structure which is monolithic, i.e. the solid structure is permanently bonded forming a continuous solid phase. In contrast, non-membrane filters are formed by fibres held in place by mechanical entanglement or other surface forces. Membrane filters can be made with narrow pore size distribution with very small pores when necessary.

The microporous membranes which may be used in the present invention may have a pore size of greater than 0.02 μm. The pore size is preferably less than 10 μm, more preferably less than 1 μm and most preferably less than 0.5 μm; specific examples of pore sizes which may be used in the present invention are pores of about 0.2 μm and pores of about 0.1 μm. The microporous filter used in the invention may be made from various materials, including polymers, metals, ceramics, glass and carbon. Typically the membrane will be formed of a polymeric material known in the art to be used in membrane filtration, such as for example polysulphones, polyethersulphones (PES), polyacrylates, polyvinylidenes such as polyvinylidene fluoride (PVDF), polytetrafluoroethylene (PTFE), cellulose, cellulose esters or co-polymers thereof. Preferably where the encapsulating material is a protein, the membrane will be selected to comprise a low protein-binding material such as a polyethersulphone or a polyvinylidene. Such microporous filters are available from Millipore Corporation (Bedford, Mass.). The membrane filter may be a membrane disc, although other forms of membrane filters are usable in the present invention

Where a liquid solution of the protein template or subunits is subjected to a microporous membrane filtration step prior to the formation of nanoparticles, a liquid solution of the protein template is first prepared, normally an aqueous solution which is then subjected to the microporous membrane filtration step. In this step, the solution is introduced to one side of the filter and filtered through the membrane. Preferably, the solution is subjected to an applied positive pressure during the filtration step. For example the applied pressure may be greater than 1 psi, for instance greater than 5 psi. Normally, the pressure will be less than 20 psi, for instance less than 15 psi. The filtrate, which comprises a solution of the protein template (or a subunit thereof) is then recovered, for use in the encapsulation of semiconductor nanoparticles in a manner which is known per se (see WO 98/22942). Where a liquid composition of preformed encapsulated nanoparticles is subjected to the microporous membrane filtration step, the nanoparticles are first formed within the protein template in a manner which is known per se (see for example WO 98/22942). A solution of the nanoparticles, preferably an aqueous solution, is then subjected to the microporous membrane filtration step. In this filtration step, the solution is introduced to one side of the filter and filtered through the membrane. Preferably, the solution is subjected to an applied positive pressure during the filtration step, for example to an applied pressure of greater than 1 psi or less than 20 psi, preferably greater than 5 psi or less than 15 psi. The filtrate, which comprises a solution of the encapsulated semiconductor nanoparticles is then recovered.

The protein encapsulant may have attached to it one or more biological ligands. Such ligands may be attached by covalent bonds. The ordinary addressee will understand that a variety of ligands such as antibodies or derivatives thereof, receptor molecules, opsonins etc. may be attached to the surface of the protein shell. Examples of biological ligands which may be attached to the protein shell are biotin and avidin.

As discussed above, the protein encapsulating material may be treated or removed from the protein-encapsulated semi-conductor nanoparticles of the second aspect of the present invention. Such treated nanoparticles, in particular, the semiconductor nanoparticle core per se and carbon coated nanoparticle cores represent further aspects of the present invention.

The nanoparticles of the present invention may find a variety of uses. For example semiconductor particles of the invention may be used in solar cells, diagnostics and biolabeling, optical fibre communication modalities such as optical modulators and amplifiers, laser diodes etc.

Solar Cells

The effectiveness with which solar cells comprising semiconductors and/or inorganic polymers absorb sunlight depends on the band-gap energy of the materials used. A typical solar cell comprises a layer of semiconductor material and optionally conducting polymers arranged between two electrodes. Typically an anti-reflective layer and a protection layer of material to protect the semiconductor particles is also included. Photons absorbed by the polymer/inorganic material generate a current which is then draw off by the adjacent electrodes. A nanoparticle of the present invention may be used to create an inorganic matrix. More particularly material of the present invention may comprise either one or a plurality of semi-conductor compounds. By tuning the size and composition of the semiconducting nanoparticles in a solar cell the available band-gap is increased thereby enabling a greater portion of the light spectrum to be absorbed. Moreover, the protein shell may facilitate uniform, high-density packing of the nanocrystals.

Biolabelling

The use of biolabels to assay for peptides, antibodies or oligonucleotides is well described, for example as illustrated in U.S. Pat. No. 6,326,144, U.S. Pat. No. 6,417,340 and U.S. Pat. No. 6,406,921, respectively. Protein encapsulated SCNM of the present invention may be conjugated to such bioligands using standard techniques. For example, amino-modified oligonucleotides may be conjugated to modified protein using sulfo-SMCC bifunctional linkers (Parak et. al. supra.). Alternatively antibodies may be conjugated to protein shells using p, p-difluoro-m,m′-dinitrophenyl sulfone, toluene diisocynate or glutaraldehyde (Wong SS supra; Bayer et.al. supra). Multiplex systems may be created in which SCNM having different emission spectra are conjugated via the protein shell to bioligands have different specificities. Fluorescence spectroscopic assays are well known in the art, for example by Lakowicz, J. R., “Topics in Fluorescence Spectroscopy”, volumes 1 to 3, New York: Plenum Press (1991). The use of an antibody-conjugated to a SCNM may readily be applied to conventional immunoassay techniques. For example, monoclonal antibodies having different specificity for target bacterial or viral species conjugated to protein templates encapsulating different SCNM, for example CdSe or CdTe, may be used to assay for particular viral or bacterial species in clinical specimens. Reference micro-organism species are bound to PVC microtitre plates in parallel with clinical specimen extracts. The antibody-SCNM are then be reacted with the wells of the microtitre plates which are then washed and analysed by a fluorescence plate spectrophotometer.

### EXAMPLES

The invention will now be illustrated by the following non-limiting examples:

### Example 1

Apoferritin Production

This example illustrates the preparation of apoferritin from horse spleen ferritin. Apoferritin was prepared from cadmium-free native horse spleen ferritin by dialysis (molecular weight cut-off of 10-14 kD) against sodium acetate solution (0.2 M) buffered at pH 5.5 under a nitrogen flow with reductive chelation using thioglycolic acid (0.3 M) to remove the ferrihydrite core. This was followed by repeated dialysis against sodium chloride solution (0.15 M) to completely remove the reduced ferrihydrite core from solution.

### Example 2

Synthesis of Cadmium Selenide-Apoferritin

Apoferritin was added to 3-([1,1-dimethyl-2-hydroxyethyl]amino)-2-hydroxypropanesulphonic acid (AMPSO)N-tris (hydroxymethyl)methyl-2-aminoethanesulphonic acid buffer, pH 8.5, to give a working solution of approximately 1.0 mg/ml. This was carefully de-aerated under nitrogen for 1 hr. and the solution maintained at a temperature of approximately 30° C. A de-aerated solution of Cd²⁺ acetate solution (0.0001 mM/mg of apoferritin) was then added to the apoferritin and stirred under positive pressure of nitrogen. The increment contained approximately 50 atoms of Cd²⁺. After approximately 1 hr an aqueous solution of sodium selenide was added in a stoichiometric amount and stirred under N₂ for 45-60 min before being dialysing against AMPSO, buffered to pH 8.5. CdSe ferritins with higher loadings were prepared by increasing the number of iterations applied in the step-wise synthesis. The samples were dialysed after each selenisation reaction.

Control experiments were conducted by reacting de-aerated solutions of Cd²⁺ acetate and sodium selenide in the absence of ferritin.

### Example 3

Synthesis of Zinc Selenide-Apoferritin

Apoferritin was added to 3-([1,1-dimethyl-2-hydroxyethyl]amino)-2-hydroxypropanesulphonic acid (AMPSO)N-tris (hydroxymethyl)methyl-2-aminoethanesulphonic acid buffer, pH 8.5, to give a working solution of approximately 1.0 mg/ml. This was carefully de-aerated under nitrogen for 1 hr and the solution maintained at a temperature of approximately 30° C. A de-aerated solution of Zinc²⁺ acetate solution (0.0001 mM/mg of apoferritin) was then added to the apoferritin and stirred under positive pressure of nitrogen. The increment contained approximately 50 atoms of Zn²⁺. After approximately 1 hr an aqueous solution of sodium selenide was added in a stoichiometric amount and stirred under N₂ for 45-60 min before being dialysing against AMPSO, buffered to pH 8.5. ZnSe ferritins with higher loadings were prepared by increasing the number of iterations applied in the step-wise synthesis. The samples were dialysed after each selenisation reaction.

Control experiments were conducted by reacting de-aerated solutions of Zn²⁺ acetate and sodium selenide in the absence of ferritin.

### Example 4

Synthesis of Zinc Sulphide-Apoferritin

Apoferritin was added to 3-([1,1-dimethyl-2-hydroxyethyl]amino)-2-hydroxypropanesulphonic acid (AMPSO)N-tris (hydroxymethyl)methyl-2-aminoethanesulphonic acid buffer, pH 8.5, to give a working solution of approximately 1.0 mg/ml. This was carefully de-aerated under nitrogen for 1 hr and the solution maintained at a temperature of approximately 30° C. A de-aerated solution of Zinc²⁺ acetate solution (0.0001 mM/mg of apoferritin) was then added to the apoferritin and stirred under positive pressure of nitrogen. The increment contained approximately 50 atoms of Cd²⁺. After approximately 1 hr an aqueous solution of sodium sulphide was added in a stoichiometric amount and stirred under N₂ for 45-60 min before being dialysing against AMPSO, buffered to pH 8.5. ZnS ferritins with higher loadings were prepared by increasing the number of iterations applied in the step-wise synthesis. The samples were dialysed after each sulphidation reaction.

Control experiments were conducted by reacting de-aerated solutions of Zn⁺ acetate and sodium sulphide in the absence of ferritin.

Characterization

Synthesised samples were, with the naked eye, visibly free from precipitate and colourless or slightly coloured; the colouration being dependent on the size of the encapsulated particles and their associated band gap energy. Characterization was in the main performed using UV-vis spectroscopy to give a measure of the absorbance band gap and hence particle size. TEM analysis of a sample produced by the method of Example 2 showed the particles to be discrete with a narrow size range of 2 nm for 100 atoms loading. Negative staining with a heavy element, uranyl acetate, showed that the protein was intact (halo-effect) and that the synthesised particles were encapsulated with the protein ferritin. Electron diffraction gave a ring pattern corresponding to CdSe wurtzite structure. EDX elemental analysis gave the corresponding elements for cadmium and selenium. Gel electrophoresis of the samples and staining for protein and cadmium showed that the metallic element to be associated with the protein with the respective bands running through the gel con-currently.

In all instances control experiments did not produce colourless or stable solutions. Examination and analysis of the control samples showed non-discrete aggregates, with a large particles size range (50-100 nm), these being several orders of magnitude larger than those encapsulated within the ferritin.

In all instances control experiments did not produce colourless or stable solutions. Examination and analysis of the control samples showed non-discrete aggregates, with a particles several orders of magnitude larger than those than those encapsulated within the ferritin.

### Example 5

Conjugation of Monoclonal Antibodies to Ferritin.

Protein-A/sepharose affinity-purified anti-HLA-A2 monoclonal antibody MA2.1 was biotinylated using conventional procedures to yield approximately four biotins per antibody molecule. The biotinylated antibody at approximately 1 mg/ml was mixed in approximately 1:1 ratios with 1 mg/ml avidinylated ferritin (Sigma) in 50 mM HEPES buffer for 1 hour at room temperature. Unbound antibody was removed by micro-centrifugation using a 300 K cut-off filter (Nano-sep® Gelman Labs).

The resulting conjugate was tested against sections of human tissue bearing the HLA-A2 antigen by conventional DAB/horse-radish peroxidase immunohistochemistry. It was found that the MA2.1:ferritin conjugate bound to a greater extent than avidinylated ferritin alone.

1. A method for the synthesis of a semiconductor nanoparticle within a protein template, wherein the semiconductor is selected from cadmium selenide, cadmium telluride, zinc selenide, zinc sulfide and zinc telluride, which process comprises forming a reaction mixture by combining in a liquid medium a cation source selected from cadmium or zinc ions and an anion source selected from sulfur, selenium or tellurium ions in the presence of a source of a protein which is capable of acting as a template for the formation of nanoparticles and maintaining said liquid medium at a temperature of at least 24° C. for a time sufficient to permit nanoparticle formation within the protein template, with the proviso that when the cation source is cadmium, the anion source is not sulfur.

2. A method in accordance with claim 1, wherein said cation source is a salt of cadmium or zinc.

3. A method in accordance with claim 2, wherein said salt comprises an acetate, nitrate or sulphate group.

4. A method in accordance with claim 1, wherein said anion source is a salt.

5. A method in accordance with claim 4, wherein said salt is a sodium salt of sulfur, selenium or tellurium.

6. A method in accordance with claim 1, wherein said anion source is selected from the group comprising hydrogen sulphide, hydrogen telluride or hydrogen selenide.

7. A method in accordance with claim 1 wherein said liquid medium is an aqueous solution.

8. A method in accordance with claim 7 wherein said aqueous solution comprises one or more water miscible solvents.

9. A method in accordance with claim 8, wherein said water miscible solvents comprise tetrahydrofuran or ethanol.

10. A method in accordance with claim 8, wherein said water miscible solvents are present in an amount of less than 25% by weight.

11. A method in accordance with claim 10, wherein said water miscible solvents are present in an amount of less than 10% by weight.

12. A method in accordance with claim 1 wherein said cation and anion sources are added in incremental quantities to an aqueous solution of said protein source.

13. A method in accordance with claim 12 wherein said cation and anion sources are added in sufficient amounts to provide 1-200 atoms of the cation and anion per protein template per iteration.

14. A method in accordance with claim 13 wherein said cation and anion sources are added in sufficient amounts to provide 20-100 atoms per protein template per iteration.

15. A method in accordance with claim 14 wherein said cation and anion sources are added in sufficient amounts to provide 50 atoms per protein template per iteration.

16. A method in accordance with claim 1 wherein said cation and anion sources are added to the reaction mixture under inert conditions.

17. A method in accordance with claim 1 wherein the stoichiometric ratio of said cation source to said anion source is no greater than two.

18. A method in accordance with claim 1, wherein said protein template is selected from the group comprising members of the ferritin family, viruses, bacteriophages, flagellar LP rings, microtubules and chaperonins.

19. A method in accordance with claim 18, wherein said member of the ferritin family is selected from the group comprising apoferritin and DPS.

20. A method in accordance with claim 19, wherein said protein template comprises apoferritin.

21. A method in accordance with claim 1 wherein said reaction mixture is maintained at a temperature not greater than 70° C.

22. A method in accordance with claim 21 wherein said reaction mixture is maintained at a temperature in the range from 25° C. to 45° C.

23. A method in accordance with claim 22 wherein said reaction mixture is maintained at a temperature of about 30° C.

24. A method in accordance with claim 1 wherein said protein template is subjected to a size fractionation step prior to its use in the synthesis of the semi-conductor nanoparticle.

25. A method in accordance with claim 1 wherein the encapsulated nanoparticle is subjected to a size fractionation step.

26. A method in accordance with claims 24 or 25 wherein said size fractionation step is a membrane filtration step.

27. A method in accordance with claim 26, wherein the pore size of the filter is in the range from about 0.02-10 μm.

28. A method in accordance with claim 26, wherein the pore size of said filter is less than about 1 μm.

29. A method in accordance with claim 28, wherein the pore size of said filter is less than about 0.5 μm.

30. A method in accordance with claim 29 wherein the pore size of said filter is not greater than about 0.21 μm.

31. A method in accordance with claim 30 wherein the pore size of said filter is about 0.1 μm.

32. A method in accordance with claim 26, wherein the membrane filter is made from a material selected from the group comprising polymeric materials, metals, ceramics, glass or carbon.

33. A method in accordance with claim 32, wherein said material comprises a polymer.

34. A method in accordance with claim 33, wherein said polymer is selected from the group comprising polysulphones, polyethersulphones (PES), polyacrylates, polyvinylidenes, polytetrafluoroethylene (PTFE), cellulose, cellulose esters or co-polymers thereof.

35. A method in accordance with claim 34, wherein said polymer comprises a polyethersulphone or a polyvinylidene.

36. A method in accordance with claim 26, wherein the solution is subjected to an applied positive pressure during the filtration step.

37. A method in accordance with claim 1, wherein the semiconductor nanoparticles vary in size by no more than 20%.

38. A method in accordance with claim 37, wherein said semiconductor nanoparticles vary in size by no more than 10%.

39. A method in accordance with claim 38, wherein said semiconductor nanoparticles vary in size by no more than 5%.

40. A method in accordance with claim 1, wherein at least 50% by weight of said nanoparticles are present as single unagglomerated particles.

41. A method in accordance with claim 40, wherein at least 70% by weight of said nanoparticles are present as single unagglomerated particles.

42. A method in accordance with claim 1, wherein said protein template comprises one or more biological ligands.

43. A method in accordance with claim 42, wherein said biological ligands are selected from the group comprising antibodies or derivatives thereof, receptor molecules, opsonins, biotin and avidin.

44. A protein-encapsulated semiconductor nanoparticle obtainable by the process of claim 1, wherein said semiconductor is selected from CdSe, CdTe, ZnSe, ZnS or ZnTe.

45. A protein-encapsulated semiconductor nanoparticle in accordance with claim 44, wherein said semi-conductor nanoparticle is CdSe or CdTe.

46. A protein-encapsulated semiconductor nanoparticle in accordance with claim 45, wherein said protein template is selected from the group comprising DPS or apoferritin.

47. A protein-encapsulated semiconductor nanoparticle in accordance with claim 44, wherein said protein template is selected from the group comprising members of the ferritin family, viruses, bacteriophages, flagellar LP rings, microtubules and chaperonins.

48. A protein-encapsulated semiconductor nanoparticle in accordance with claim 47, wherein said member of the ferritin family is selected from the group comprising apoferritin and DPS.

49. A protein-encapsulated semiconductor nanoparticle in accordance with claim 48, wherein said protein comprises apoferritin.

50. A protein-encapsulated semi-conductor nanoparticle in accordance with claim 44, wherein said semiconductor nanoparticle has a diameter of up to about 15 nm.

51. A protein-encapsulated semi-conductor nanoparticle in accordance with claim 44, wherein said semiconductor nanoparticle varies in size by no more than 20%.

52. A protein-encapsulated semi-conductor nanoparticle in accordance with claim 51, wherein said semiconductor nanoparticle varies in size by no more than 10%.

53. A protein-encapsulated semi-conductor nanoparticle in accordance with claim 52, wherein said semiconductor nanoparticle varies in size by no more than 5%.

54. A protein-encapsulated semi-conductor nanoparticle in accordance with claim 44, wherein said protein template comprises one or more biological ligands.

55. A protein-encapsulated semi-conductor nanoparticle in accordance with claim 54, wherein said biological ligands are selected from the group comprising antibodies or derivatives thereof, receptor molecules, opsonins, biotin or avidin.

56. A semiconductor nanoparticle selected from CdSe, CdTe, ZnSe, ZnS or ZnTe obtained by treating or removing the protein encapsulating material from the protein-encapsulated semiconductor nanoparticles of claim 44.

57. The semiconductor nanoparticle of claim 56, wherein said protein encapsulating material is removed by enzymatic degradation.

58. The semiconductor nanoparticle of claim 57, wherein the enzyme is a protease.

59. The semiconductor nanoparticle of claim 56, wherein the protein encapsulating material is removed by pH denaturation.

60. The semiconductor nanoparticle of claim 59, wherein said pH denaturation is effected by adjusting the pH of the solution to a value below about 4.0 or above about 9.0.

61. The semiconductor nanoparticle of claim 56, wherein the size of the semiconductor nanoparticle varies by no more than about 20%.

62. The semiconductor nanoparticle of claim 61, wherein the size of the semiconductor nanoparticle varies by no more than about 10%.

63. The semiconductor nanoparticle of claim 62, wherein the size of the semiconductor nanoparticle varies by no more than about 5%.

64. The semiconductor nanoparticle of claim 56, wherein said protein encapsulating material is treated by carbonisation.

65. The semiconductor nanoparticle of claim 56, wherein said protein encapsulating material is treated by the attachment of biological ligands.

66. The semiconductor nanoparticle of claim 65, wherein said biological ligands are selected from the group comprising antibodies or derivatives thereof, receptor molecules, opsonins, biotin and avidin.

67. A composition of protein-encapsulated semi-conductor nanoparticles, wherein said semiconductor is selected from CdSe, CdTe, ZnSe, ZnS or ZnTe.

68. A composition of protein-encapsulated semi-conductor nanoparticles, wherein said semiconductor is selected from CdSe or CdTe.

69. A composition in accordance with claim 68, wherein said protein comprises DPS or apoferritin.

70. A composition in accordance with claim 67, wherein said protein is selected from the group comprising members of the ferritin family, viruses, bacteriophages, flagellar LP rings, microtubules and chaperonins.

71. A composition in accordance with claim 70, wherein said member of the ferritin family is selected from apoferritin and DPS.

72. A composition in accordance with claim 71, wherein said protein comprises apoferritin.

73. A composition in accordance with claim 67, wherein at least 50% by weight of said protein-encapsulated semi-conductor nanoparticles are present as single unagglomerated particles.

74. A composition in accordance with claim 73, wherein at least 70% by weight of said protein-encapsulated semi-conductor nanoparticles are present as single unagglomerated particles.

75. A composition in accordance with claim 67, wherein said semi-conductor nanoparticles vary in size by no more than 20%.

76. A composition in accordance with claim 75, wherein said semi-conductor nanoparticles vary in size by no more than 10%.

77. A composition in accordance with claim 76, wherein said semi-conductor nanoparticles vary in size by no more than 5%.

78. A composition in accordance with claim 67 wherein said protein comprises one or more biological ligands.

79. A composition in accordance with claim 78, wherein said biological ligands are selected from the group comprising antibodies or derivatives thereof, receptor molecules, opsonins and biotin or avidin.

80. A composition in accordance with claim 67 wherein said protein is carbonised.

81. Use of a protein encapsulated semiconductor nanoparticle in accordance with claim 67 in a solar cell.

82. Use of a protein encapsulated semiconductor nanoparticle in accordance with claim 67 in immunoassay techniques.
